(19) United States
(12) Patent Application Publication
(10) Pub. No.: US 2024/0210390 A1
(43) Pub. Date: Jun. 27, 2024

(54) SURFACE-EFFECT SENSOR AND FORMULATION

(71) Applicant: CALYX, INC., Berkeley, CA (US)

(72) Inventors: Benson FAN, Berkeley, CA (US); Po Jui CHIU, Berkeley, CA (US); I-Ting CHEN, Berkeley, CA (US)

(73) Assignee: CALYX, INC., Berkeley, CA (US)

(21) Appl. No.: 18/557,656

(22) PCT Filed: Apr. 29, 2022
(86) PCT No.: PCT/US2022/027031

§ 371 Date: Oct. 27, 2023

Related U.S. Application Data
(60) Provisional application No. 63/181,876, filed on Apr. 29, 2021.

(51) Int. Cl. G01N 33/543; G01N 29/02; G01N 29/036

(52) U.S. Cl. CPC G01N 33/5438; G01N 29/022; G01N 29/036; G01N 2291/0255; G01N 2291/0256; G01N 2291/0423; G01N 2291/0426

(57) ABSTRACT

An area on a sensor chip (700) has a coating that contains fiber bundles (622) including a phage and is bioengineered to interact with a target analyte. A quartz crystal microbalance (QCM) or surface acoustic wave (SAW) sensing circuit, which may be connected to or integrated into the sensor chip (700), measures effects that result from interaction of the coating with the target analyte. Spin coating or spray coating may produce a coating suitable for QCM or SAM sensing techniques.

### BACKGROUND

Sensing devices are needed that can provide selectivity, sensitivity, and real-time point detection, multi-analyte assessment, ease-of-use, and low cost. Costs is a particular problem for high capability sensors because current manufacturing process for high capability sensors generally require high system complexity, extensive chemical synthesis and fabrication, and/or integration of additional sensor components such as sample preparation chambers.

The best current techniques for small-molecule gas sensing include gas chromatography-mass spectrometry (GC-MS). GC-MS machines, however, are large and expensive. Optical sensing systems based on Raman or absorption spectroscopy are also available but have selectivity that can be poor under realistic operating conditions and currently have sensitivity in the 1-ppm range, which is insufficient for some applications.

Furthermore, while Raman or absorption spectroscopic systems may be effective at detecting smaller molecules such as carbon dioxide (CO₂) or oxides of nitrogen (NO_{X}), such sensing systems are not as effective for more complex molecules, whose vibrational or rotational spectra are difficult to isolate, especially in mixtures including typical background molecules.

U.S. Pat. App. Pub. No. 2016/0312262, entitled “BIOMIMETIC VIRUS-BASED COLORIMETRIC SENSORS,” describes chemical sensing using a colorimetric detection layer that undergoes a color change upon interaction with an analyte of interest. The colorimetric detection layer may include self-assembled fiber bundles, where at least a fraction of the fiber bundles changes conformation (and therefore change color) upon interaction with the target analyte. Self-assembled fiber bundles specific to target analytes may be engineered using known techniques such as described in U.S. Pat. App. Pub. No. 2011/0311490, entitled “Recombinant Bacteriophages Useful for Tissue Engineering.” U.S. Pat. Nos. 5,223,409 and 5,571,698 to Ladner et al., entitled “Directed Evolution of Novel Binding Proteins,” also describe the use of filamentous virus in so-called directed evolution. Biopanning is an affinity selection technique for selecting peptides that bind to a given target, and in a free database commonly known as BDB stores combinatorial peptide libraries. These systems may require use of chemical layers or highly organized structures that are expensive to manufacture and may require complex color sensing systems that can be difficult to inexpensively miniaturize.

Current sensor systems are not ideal in terms of ease-of-use, cost, and precision. Current sensing technology need improvements to provide low costs and high sensitivity to targeted analytes or to a range of analytes.

### BRIEF DESCRIPTION OF THE DRAWINGS

FIG. 1 is a block diagram of a sensor system in accordance with an example of the present disclosure.

FIG. 2 is a block diagram of a sensor system providing analyte measurements or detection based on changes the analyte causes in the color of a sensing layer in accordance with an example of the present disclosure.

FIG. 3 is a block diagram of a sensor system in accordance with an example of the present disclosure providing analyte measurements or detection based on surface effects that interactions of the analyte with a sensing layer causes.

FIG. 4 is a block diagram of a testing or calibration system for sensor chips in accordance with an example of the present disclosure.

FIG. 5 shows a plot of the response to changing concentrations of a target analyte in a sensor chip in accordance with an example of the present disclosure.

FIG. 6 schematically illustrates structure in a sensing layer in accordance with an example of the present disclosure.

FIG. 7 shows a plan view of a sensor chip in accordance with an example of the present disclosure.

FIG. 8 is a flow diagram of a process for fabricating a sensing layer in accordance with an example of the present disclosure.

FIG. 9 illustrates a hierarchy of structures in a compact or miniature sensor capable of sensing one or more specific chemical compounds using bioengineered phage technology.

FIG. 10 shows a block diagram of sensor in accordance with an example of the present disclosure including an array of different sensor chips targeting different analytes.

The drawings illustrate examples for the purpose of explanation and are not of the invention itself. Use of the same reference symbols in different figures indicates similar or identical items.

### DETAILED DESCRIPTION

Miniaturized, smart sensor systems as disclosed herein may be cost effective and may provide real-time results with high sensitivity and selectivity, particularly for detecting small molecule chemical vapors. A sensor may be employed, for example, in energy, defense, and health applications to detect or measure a concentration of a target analyte. Sensing systems and processes for manufacturing sensor chips as disclosed herein may provide selectivity and sensitivity to target analytes or to a target group of analytes in sensors at a cost that permits widespread use in applications where such sensors were previously cost prohibited. A formulation or fabrication process as disclosed herein can provide a cost-effective biomimetic sensing layer that a target analyte or group of analytes activates and a sensing system that can detect physical characteristics or surface effects that the target analyte or group of analytes changes or causes. The changed characteristics are not restricted to color but can include acoustic characteristics, which may be measured using electronics that can be integrated into or separate from a substrate on which the biomimetic sensing layer is formed. When acoustic characteristics are most important, fabrication techniques such as spin coating or spray coating can produce a sensing layer that is highly effective at interacting with the target analyte but may not have the highly ordered structure needed for the consistent color changes necessary in colorimetric sensors.

U.S. Pat. App. Pub. No. 2016/0312262, entitled “BIOMIMETIC VIRUS-BASED COLORIMETRIC SENSORS,” describes chemical sensing using a colorimetric detection layer that undergoes a color change upon interaction with an analyte of interest, e.g., the target analyte. In accordance with an aspect of the present disclosure, a sensor technology using fiber bundles is not limited to colorimetric sensing. The same fundamental mechanism of the fiber interaction with the chemical target or analyte can be transformed into a sensing system using microelectromechanical systems (MEMS), enabling even further miniaturization and power efficiency. In a MEMS-based sensor, sensor formulations for a sensing layer or film can be deposited on a MEMS substrate in the same way sensor formulations may deposited for a colorimetric sensor or using techniques that may not provide the highly ordered structure that provides consistent color changes. In a biomimetic sensing layer or film, engineered phage materials may be aligned in a periodic pattern with a specific orientation or may be more randomly arranged. The resulting sensing layer or film changes characteristics, e.g., lattice parameters or density, when the target chemical or analyte is presented at a sample, e.g., air, another gas, or a liquid, that interacts with the sensing layer. This change in the sensing layer or film can be measured as a shift in the oscillation frequency on a MEMS substrate on which the sensing layer is formed, and the size of frequency shift, e.g., in acoustic waves traversing the MEMS substrate or a resonance in the MEMS substrate, can be correlated in real-time to the concentration of the target chemical.

In accordance with a further aspect of the present disclosure, a sensor employs an analyte-specific formulation or surface treatment (or sensing film, layer, or coating) in a sensor such as a quartz crystal microbalance (QCM) sensor or a surface acoustic wave (SAW) sensor that measures surface effects of the surface treatment. The structure of the surface treatment changes depending on the concentration of analytes presented at the surface treatment. Sensor electronics may measure or detect the analyte-induced change, for example, by mass change, frequency (oscillation), electrical characteristics (conductivity, resistance), or other means and the measured change may be converted to a measurement of analyte concentration.

### Example Sensors

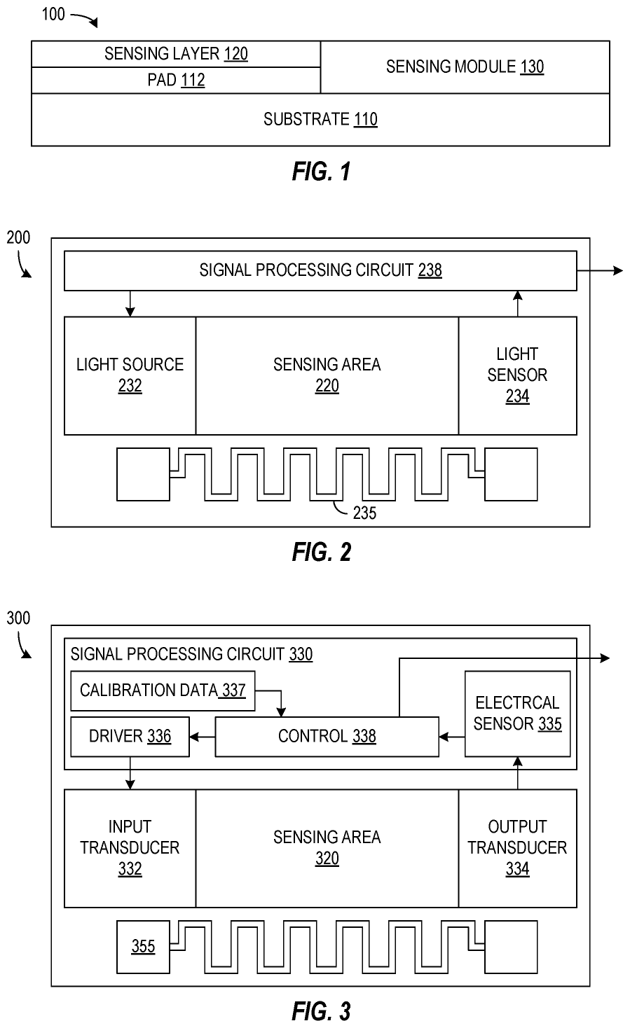

FIG. 1 is a block diagram of a sensor 100 in accordance with one example of the present disclosure. Sensor 100 includes a substrate 110, a sensing layer 120, and a sensing module 130. Sensing layer 120 may be a surface treatment, film, coating, or other layer that may be deposited on or applied to substrate 110 on a portion of substrate 110. For example, substrate 110 may include a pad 112, e.g., a gold pad, that may better suited than the underlying material of substrate 110 to provide a surface for the treatment that forms sensing layer 120 on the surface of pad 112. Sensing module 130 may include electrical, photoelectric, optical, or electromechanical elements, all or portions of which may be integrated on substrate 110 or may be one or more separate devices connected for measurement of one or more characteristics or surface effects of sensing layer 120. Sensing layer 120 contains a network of fiber bundles chosen so that at least a fraction of the fiber bundles undergoes a change from one state to another state when interacting with an analyte of interest.

FIG. 2 illustrates a more specific example of a sensor 200 in accordance with an example of the present disclosure. Sensor 200 is a colorimetric sensor capable of detecting the presence or measuring concentrations of a target analyte in a sample, e.g., a gas or air sample, introduced to a sensing area 220. A sensing module of sensor 200 may include sample control systems 235 that monitor and control conditions during measurements. Control systems 235 may include one or more heater or heating element to control the temperature of sensing area 220 or the sample or include a humidity control system to control the humidity of the sample in sensing area 220. Sensing area 220 contains a sensing layer as described further herein, which may be a network of fiber bundles, at least a fraction of which change when interacting with the target analyte such that the sensing layer in sensing area 220 changes the color. The sensing module of sensor 200 further includes a light source 232 positioned and operable to illuminate sensing area 220 and a light sensor 234 adapted to sense light reflected from sensing area 220. Light sensor 234 may particularly detect spectral content of the reflected light or sense light at a wavelength associated with the change that the target analyte causes in the sensing layer in sensing area 220. A signal processing circuit 238 may convert the signal(s) from light sensor(s) 234 into a measurement of concentration of the target analyte in the sample.

FIG. 3 illustrates another example of a sensor 300 in accordance with an example of the present disclosure. Sensor 300 detects the presence or measures concentrations of a target analyte in a sample in a sensing area 320 by sensing changes a sensing layer in a sensing area 320 causes to acoustic characteristics of sensor 300. The sensing module of sensor 300, like the sensing module of sensor 200, may include sample control systems 335 that monitor and control conditions such as temperature and humidity of the sample or sensing area 320 during measurements. Sample control systems 335 may include one or more heater or heating element to control the temperature of sensing area 320 or the sample or include a humidity control system to control the humidity of the sample in sensing area 320. Sensing area 320 contains a sensing layer as described further herein, which may be a network of fiber bundles, at least a fraction of which change when interacting with the target analyte such that the sensing layer in sensing area 320 changes density or other acoustic characteristics in sensor 300 and may cause surface effects that more widely change characteristics in sensor 300. The sensing module of sensor 300 further includes an input transducer 332 and an output transducer 334. Input transducer 332 may be positioned at one edge of sensing area 320, so that an electric drive circuit 336 can apply a drive signal causing input transducer 332 to create acoustic or sound wave at the edge of sensing area 320. Output transducer 334 may be at an edge of sensing area 320 opposite from input transducer 332 and operates to convert acoustic vibrations to a raw electrical measurement signal.

An electrical sensor 335 in signal processing circuit 330 measures an electrical signal from output transducer 334 or otherwise use output transducer 334 to generate a raw measurement signal. The raw measurement signal may, for example, be used to determine a phase difference between the drive signal and the electrical signal induced in output transducer, a resonant frequency or a change in a resonant frequency in sensor 300, or a signal propagation time for acoustic waves in sensing area 320. For a QCM measurement technique, electrical sensor 335 measures a resonance frequency (or a change in the resonance frequency) of a quartz structure over which the sensing layer is formed. The resonant frequency may be measured by driving input transducer 332 to create acoustic vibrations, shutting off the drive signal, and measuring the frequency that become dominant in the decay of the acoustic vibration. For an SAW measurement technique, surface acoustic waves at the surface of the sensing area 320 where the sensing layer resides can change the propagation time of acoustic waves or the frequency of the raw electrical signal from output transducer 334. With any of these sensing techniques, when a quantity of the target analyte held in the sensing layer changes as a result of a change in the concentration target analyte in the sample in sensing area 320, the raw signal extracted from output transducer 334 changes, and control circuit 338, e.g., a microcontroller, can use calibration data 337 to convert the raw signal or the change in the raw signal from output transducer 334 and electrical sensor 335 into a measurement signal indicating the measured concentration of the target analyte.

Sensors 200 and 300 of FIGS. 2 and 3 are examples of sensors using a sensing layer as disclosed herein. Other sensors employing sensing layers as disclosed herein may employ a variety of sensing techniques, for example, by measurement of mass change, frequency (oscillation), electrical characteristics (conductivity, resistance), or other characteristics of a sensing layer or a structure that the sensing layer affects.

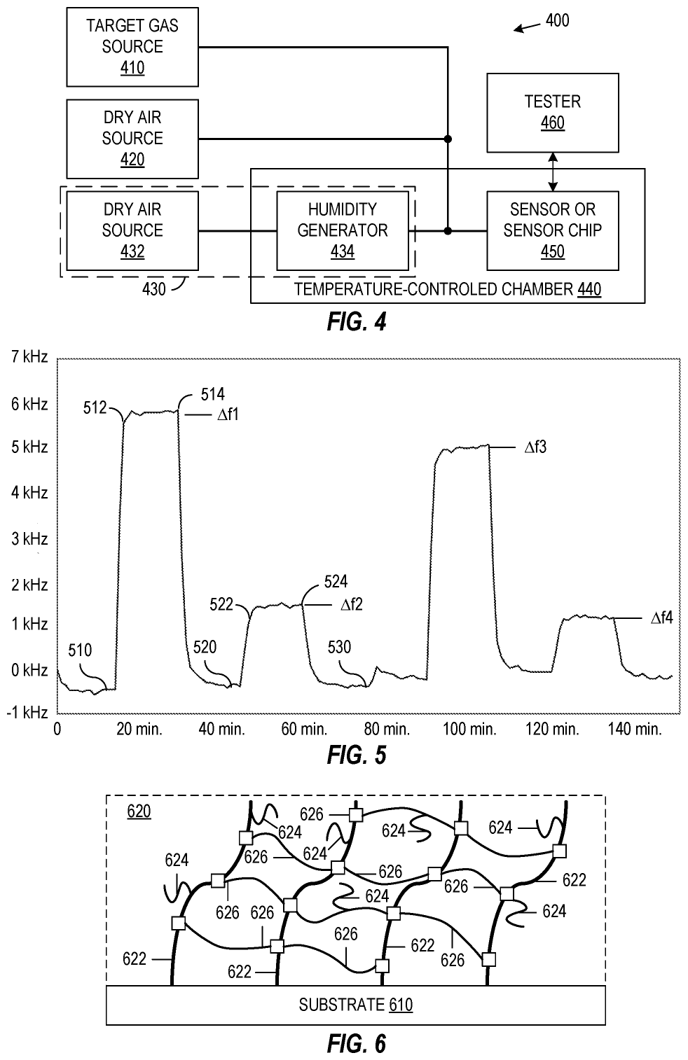

FIG. 4 shows a block diagram of a testing or calibration system 400 for a sensor chip 450 in accordance with an example of the present disclosure. (Sensor chip 450 may constitute at least a portion of sensor 300 of FIG. 3 and may or may not include active electronics such as signal processing circuit 330.) System 400 uses a source 410 of a target gas, i.e., a gas consisting of or containing the target analyte to be sensed, a source 420 of dry air, and a source 430 of humid air. Humid air source 430 may include a dry air source 432 and a humidity generator 434 capable of adding a controlled amount of humidity to an air flow from dry air source 432. A calibration or testing process for a sensor chip may operate sources 410, 420, and 430 to produce a gas sample or mixture having a controlled concentration of the target gas and a controlled humidity. The sample gas flows into or fills a temperature-controlled chamber 440 containing the sensor chip 450 under test. For the test or calibration, sources 410 and 420 can be controlled to introduce a desired concentration of the target gas into chamber 440, at which point a tester 460 can measure the response of sensor chip 450 to calibrate how the response changes with concentrations of the target analyte at one or more specified humidity. The results of tests at multiple concentrations of the target gas and optionally at multiple humidity may be used to calibrate a sensor, e.g., to determine calibration data 337 needed for operation of sensor 300 of FIG. 3 using the tested sensor chip 450.

FIG. 5 shows a plot of resonance frequency shifts resulting from varying concentrations of a target analyte during testing of a sensor chip, e.g., using test or calibration system 400 on sensor chip 450 of FIG. 4. In general, different formulations for a sensing layer and different geometries or examples of senor chips may provide differing responses to the same concentration of a target analyte such as ammonia (NH₃) or carbon dioxide (CO₂). Sensor chips accordingly need testing or calibration, so that a sensor, during normal operation, can convert a response of the sensor chip into a measurement signal that indicates the detected concentration of the target analyte.

The plot of FIG. 5 includes a background period 510 when the target analyte is not present in the sample interacting with the sensor chip. When no target analyte is present, a resulting frequency shift should be at a background level 510, e.g., zero other than signal noise. When the calibration system introduces a known or controlled concentration C1 of the target analyte to the sample, a change 512 in the frequency shift begins and continues until the frequency shift plateaus, e.g., until the amount of the target analyte bound or held in the sensing layer reaches equilibrium with the concentration of the analyte in the sample. The level Δf1 of the equilibrium plateau corresponds to the known concentration C1. When the flow of the target analyte cuts off, a change 525 in the frequency shift occurs and continues until the sample and the sensing layer no longer contains the target analyte and the frequency shift again reaches the background level 520. When the calibration system introduces another known or controlled concentration C2 of the target analyte to the sample, another change 522 in the frequency shift begins and continues until the frequency shift plateaus at another level Δf2, which corresponds to the known concentration C2. A calibration process may continue to track changes in the measured frequency shifts, e.g., Δf3 and Δf4, that different concentrations of the target analyte cause, and the measured frequency shifts can be used to construction calibration data for a map, e.g., a lookup table or a function, that maps frequency shifts to concentrations of the target gas.

### Sensing Layer

Sensing layers used in sensors such as illustrated in FIGS. 1, 2, and 3 may be thin film layers that change characteristics in response to changes in the concentration of target chemicals or analytes to which the sensing layers are exposed. A fabrication process for a sensing layer can create chemical structures organized as illustrated by layer 620 shown in FIG. 6. Layer 620 includes fibers, fiber bundles, or hygroscopic polymers 620 that may be or contain biomolecules including biomolecular macromolecules and oligomers, including viruses, phages, polypeptides, polysaccharides, and nucleic acids. Fiber bundles 620 may include naturally occurring organic or biological materials as well as synthetic materials and genetically engineered materials in blended and composite arrangements. In one example, fiber bundles 620 includes biomolecules such as M13 phage bundles, and in some cases, the M13 phage can display various peptide chains that bind an analyte of interest on a portion or all of its 2,700 copies of major coat proteins. In another example, sensing layer 620 may contain long chain polymer networks such as polyethylene. In some cases, the long polymer network chain or the M13 phage 620 can act as a scaffold with cross linkers 626 interconnecting fiber bundles 622. The scaffold structure may have peptides, metals, or metallic organic complexes, which may be held in or attached to the scaffold structure. In this case, the peptides, metals, or the metallic organic complexes may be receptors 624 responsible for the sensing, and the scaffold will act to provide stability, the increased surface area, and protection of the core mA variety of detectable layer fiber structures can be made from elongated structures or fiber bundles.

Exemplary fiber bundles 622 can include aligned, crosslinked, rod-like particles as building blocks. Exemplary particles for forming fiber bundles 622 may have a cross sectional diameter of about 5 nm to about 20 nm, and a length of about 60 nm to about 6,000 nm. More particularly, the length can be about 250 nm to about 1,000 nm. In various embodiments, the particles have a cross sectional diameter in the micrometer range, e.g., at least about 1 μm, 5 μm, 10 μm, 100 μm, 250 μm, or greater.

Fiber bundles 620 may contain most any material so long as fibers can be prepared. In general, virus particles, which are long and filamentous structures, can be used. See, e.g., Genetically Engineered Viruses, Christopher Ring (Ed.), Bios Scientific, 2001. Virus particles able to function as flexible rods can be used. Besides virus particles, other examples that composes of the fiber bundle include polymer and polymeric fibers.

One example of the present disclosure uses virus particles that are not genetically engineered. Alternatively, a genetically engineered virus may also achieve desirable properties. For example, viruses can be used that have been subjected to biopanning so that the virus particles specifically bind to analytes or receptors that were the target of the biopanning. The viruses can be converted to fiber or bundle form with or without a conjugate moiety. Use of filamentous virus in so called directed evolution or biopanning is further described in the patent literature including, for example, U.S. Pat. Nos. 5,223,409 and 5,571,698 to Ladner et al. (“Directed Evolution of Novel Binding Proteins”).

A virus particle in a sensing layer can have a size and dimensions such that the particle is elongated. For example, fibrous virus material can include aligned, crosslinked, rod-like particles, and each virus particles may have a cross sectional diameter of about 5 nm to about 20 nm, and a length of about 60 nm to about 6,000 nm. More particularly, the length of each virus particle may be about 250 nm to about 1,000 nm.

A sensing layer can contain mixtures of two or more kinds of viruses and may include a mixture of virus particles with non-virus materials.

The term “virus” as used herein includes both phages and animal viruses, and a “virus particle” as used herein includes all or a portion of a virus including at least the capsid. A virus particle may, for example, include an entire virus or one or more portions of a virus including at least the virus capsid. Entire viruses can include a nucleic acid genome, a capsid, and may optionally include an envelope. Viruses in a sensing layer may further include both native and heterologous amino acid oligomers, such as cell adhesion factors. The nucleic acid genome may be either a native genome or an engineered genome.

A virus particle generally has a native structure in which the peptide and nucleic acid portions of the virus have specific arrangements. In various examples, the native structure may be preserved when the virus is incorporated into a fiber bundle. The virus and/or nucleic acids may be replicated after being fabricated into a fiber form. If during fiber formation, viral re-infectivity is lost, information may still be stored, programmed, propagated, and addressable through proteins and engineered nucleic acids, including DNA oligomers, in the virus fiber.

Exemplary viruses of use in a sensing layer include those having an expressed amino acid oligomer as a specific binding site. Amino acid oligomers can include any sequence of amino acids whether native to a virus or heterologous. Amino acid oligomers may be any length and may include non-amino acid components. Oligomers having about 5 to about 100, and more particularly, about 5 to about 30 amino acid units as specific binding site can be used. Non-amino acid components include, but are not limited to, sugars, lipids, drugs, enzymes, or inorganic molecules, including electronic, semiconducting, magnetic, and optical materials.

Sensing layers may use a wide variety of virus fibers. The fibers in a sensing layer may include only viruses of a single type or may include multiple types of viruses. Exemplary virus particles may be helical viruses. Examples of helical viruses include, but are not limited to, tobacco mosaic virus (TMV), phage pf1, phage fd1, CTX phage, and phage M13. These viruses are generally rod-shaped and may be rigid or flexible. One of skill in the art may select viruses for a sensing layer depending on the intended use and properties of the desired fiber and sensing layer.

The viruses used in some examples of sensing layers may be selected or engineered to express one or more peptide sequences including amino acid oligomers on the surface of the viruses. The amino acid oligomers may be native to the virus or heterologous sequences derived from other organisms or engineered to meet specific needs. Expression of amino acid oligomers allows engineering of the viruses forming the fibers for specific applications. For example, engineered fibers may contain amino acid oligomers that recognize a particular analyte of interest or class of analytes of interest. In various embodiments, the expressed amino acid oligomer binds and detects an organic molecule, such as an explosive, a biological warfare agent, or any other target analyte or analytes.

A genetically engineered bacteriophage in an example of the present disclosure may be a recombinant M13 bacteriophage including one or more recombinant phage coat protein that includes an amino acid sequence capable of binding the analyte of interest, e.g., the target analyte. The recombinant phage coat protein may be a recombinant pIIII. pIX, or pVIII. The signal peptide may be a peptide of any suitable length that does not interfere with the self-assembly of the M13 phage into the phage bundle structure.

One skilled in the art may employ known techniques known to make the recombinant phages with the appropriate peptide sequence. Examples of methods for making the genetically engineered bacteriophage are described in U.S. Pat. App. Pub. No. 2011/0311490 and International Pat. App. No. PCT/US2009/038449. Examples of methods for making the genetically engineered bacteriophage or amino acid sequences that bind TNT and/or DNT are described in U.S. Pat. App. Pub. No. 2012/0108450 and International Pat. App. No. PCT/US2008/060260.

In some examples disclosed herein, a sensing layer employs M13 phage that display various peptides that bind an analyte of interest on at least 10%, 20%, 30%, 40%, 50%, 60%, 70%, 80%, 90%, or all 2,700 copies of its major coat proteins. The M13 phage is a bacterial virus composed of a single-stranded DNA encapsulated by various major and minor coat proteins. It has a long-rod filament shape that is approximately 880 nm long and 6.6 nm wide. Through genetic modification, all 2700 copies of the pVIII major coat protein, which covers most of the phage surface (>98%), can display short (having fewer than 8 residues) peptide signaling molecules.

The M13 phage has several properties that make it well suited for use in a sensing layer. M13 phage is non-lytic, producing little cell debris during amplification and simplifying the amplification and purification processes. Therefore, mass amplification of the virus can be easily realized through its infection of E. coli cells, resulting in a monodisperse population of the phage. Due to their monodispersity and long-rod shape, M13 phage can self-assemble and have been extensively studied as highly organized liquid crystalline systems. The concentration of the virus suspension, ionic strength of the solution, and externally applied force fields may be used to modulate virus organization in these systems and have previously been optimized for the construction of one-, two-, and three-dimensional phage-based materials. In addition, through the insertion of random gene sequences into the phage genome, a large combinatorial library can be displayed on the phage major and minor coat proteins.

The large surface area of the recombinant M13 phage as disclosed herein and its ability to present ligands or analyte of interest in high densities make it useful for sensing layers. An engineered M13 phage has the potential for presenting a very high ligand density of ˜1.5×1013 epitopes/cm2 (3.3 nm radius, 880 nm length, 2700 pVIII units/phage).

A recombinant nucleic acid encoding a recombinant M13 bacteriophage genome can be used for the replication of the M13 phage, and be a replicon in a suitable microorganism, such as bacterial host cell, such as E. coli. Nucleic acid sequences and methods of maintaining and replicating such replicons are well known to those skilled in the art.

The recombinant phages have a short peptide motif of amino acids that is displayed on a coat protein of M13 phage. The coat protein can be pIII, pVIII, and/or pIX. In some examples, the peptide is 1-50, 1-25, 1-13, or 1-8 amino acid residues long. One skilled in the art can create partial libraries that contained randomized framing amino acids around the sequence of interest, before could successfully display the desired sequences, that also accommodated phage requirements for replication and packaging by bacteria.

Peptide sequences, which form receptors that bind to TNT and/or DNT, are taught in U.S. Pat. App. Pub. No. 2012/0108450 and International Patent Application No. PCT/US2008/060260.

Some references teach the engineering of viruses to express amino acid oligomers and may be used to assist in processes disclosed herein. For example, U.S. Pat. No. 5,403,484 by Ladner et al discloses the selection and expression of heterologous binding domains on the surface of viruses. U.S. Pat. No. 5,766,905 by Studier et al discloses a display vector comprising DNA encoding at least a portion of capsid protein followed by a cloning site for insertion of a foreign DNA sequence. The compositions described are useful in producing a virus displaying a protein or peptide of interest. U.S. Pat. No. 5,885,808 by Spooner et al discloses an adenovirus and method of modifying an adenovirus with a modified cell-binding moiety. U.S. Pat. No. 6,261,554 by Valerio et al shows an engineered gene delivery vehicle comprising a gene of interest and a viral capsid or envelope carrying a member of a specific binding pair. U.S. Pat. App. Pub. No. 2001/0019820 by Li shows viruses engineered to express ligands on their surfaces for the detection of molecules, such as polypeptides, cells, receptors, and channel proteins.

### Conjugation

The fibers used in sensing layers can be conjugated with a conjugate material altering the properties of the sensing layer, e.g., having an affinity for an analyte of interest. The conjugate material is not particularly limited. In general, the conjugate material may be selected for a particular application or target analyte. The conjugate material can be conjugated to the fibers by being subjected to viral biopanning against the conjugate material, and then the conjugate material is specifically bound to the fiber particle by, for example, a surface treatment. Conjugate material can be preformed and then bound to the fibers or can be directly formed or nucleated on the fibers. The fibers can act as a catalyst for formation of or biomineralization of the conjugate material on the fibers.

Examples of general types of conjugate materials include protein, peptide, nucleic acid, DNA, RNA, oligonucleotide, drugs, enzymes, porphyrins, metallic porphyrins, polymer nanoparticles, and metal nanoparticles. Conjugate molecules can be inorganic, organic, particulate, nanoparticulate, small molecule, single crystalline, polycrystalline, amorphous, metallic, magnetic, semiconductor, polymeric, block copolymer, functional polymer, conducting polymeric, light-emitting, phosphorescent, organic magnet, chromophore, and fluorescent materials.

The conjugate material can be directly bound to the fibers or can be linked to the fibers by an intermediate linking moiety which can both bind to the fibers and the conjugate material.

### Substrate

A substrate for a sensor as disclosed herein needs to provide a mechanical support structure and a surface on which the sensing layer may be deposited or formed. The substrate may otherwise be made of practically any physiochemically stable material. The substrates can be either rigid or flexible and can be either optically transparent or optically opaque. Substrates can be electrical insulators (e.g., quartz substrates), conductors (e.g., gold substrates), or semiconductors (e.g., silicon substrates). Further, the substrates can be substantially impermeable to liquids, vapors and/or gases or, alternatively, the substrates can be permeable to one or more of these classes of materials. Exemplary substrate materials include, but are not limited to, inorganic crystals, inorganic glasses, inorganic oxides, metals, organic polymers, and combinations thereof.

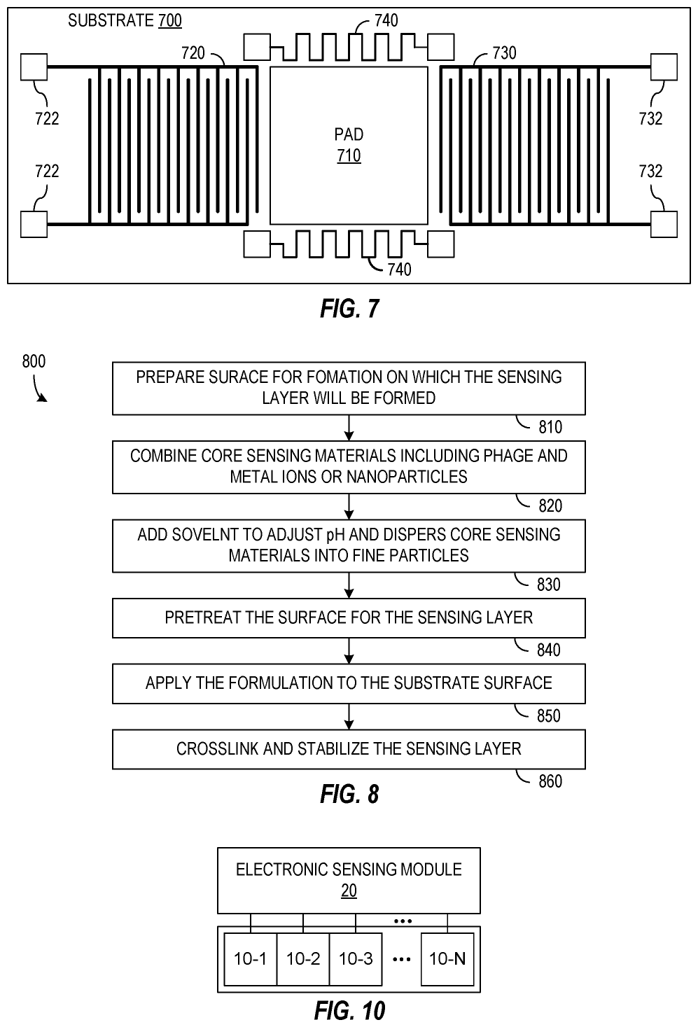

FIG. 7 shows a plan view of a substrate 700 in accordance with an example of the present disclosure. Substrate 700 may, for example, be a semiconductor or insulator substrate that includes structure including a pad 710, a pair of interdigitated electrodes 720 on one side of pad 710, another pair of interdigitated electrodes 730 an opposing side of pad 710, and one or more resistive heating elements 740 fabricated in or on substrate 700 using deposition, masking, and etching processes well known in wafer processing. Substrate 700 may be a MEMS substrate in that interdigitated electrode pairs 720 and 730 may interact with underlying piezoelectric material to convert an input electrical signal into movement of vibrations in substrate 700 or convert movement of vibrations into an output electrical signal.

Pad 710, e.g., a gold or quartz pad, provides the surface suitable for formation of a sensing layer as described herein, and the material of substate 700 and particularly of pad 710 should be non-reactive towards the constituents of the mesogenic sensing layer. Interdigitated electrodes 720 have terminals 722 that may be connected to a drive circuit (not shown) that may drive electrodes 720 with an oscillating electrical signal that creates vibrations in substrate 700.

Substrate 700 or a pad (not shown) under electrodes 720 may include a material such as quartz that exhibits a piezoelectric effect that produces or amplifies vibrations in response to the changing electric fields from electrodes 720. The vibrations propagate as acoustic waves according to the structure of substrate 700 and particularly according to the characteristics of the sensing layer on pad 710. Acoustic vibrations of interdigitated electrodes 730 thereby changing the capacitance of electrodes 730 or producing a piezoelectric voltage across electrodes 730, and a sensing circuit may be connected to generate and electrical signal that oscillates with the change in capacitance or the piezoelectrically induced voltage. As disclosed herein, changes in characteristics of the sensing layer on pad 710 can be determined from a comparison of phases of the drive signal and the measurement signal, measurement of a resonance frequency (or changes in a resonant frequency) of structure including the sensing layer on pad 710, or measurement of surface waves at a surface of the sensing layer on pad 710.

### Inorganic Crystal and Glasses

Inorganic crystals and inorganic glasses that are appropriate for substrate materials include, for example, LiF, NaF, NaCl, KBr, KI, CaF₂, MgF₂, HgF₂, BN, AsS₃. ZnS, Si₃N₄ and the like. The crystals and glasses can be prepared by art standard techniques. See, for example, Goodman, C. H. L., Crystal Growth Theory and Techniques, Plenum Press, New York 1974. Alternatively, the crystals can be purchased commercially (e.g., from Fischer Scientific). The crystals can be the sole component of the substrate, or the crystals can be coated with one or more additional substrate components. Thus, examples in accordance with the current disclosure may utilize crystal substrates coated with, for example, one or more metal films or a metal film and an organic polymer. Additionally, a crystal can constitute a portion of a substrate which contacts another portion of the substrate made of a different material, or a different physical form (e.g., a glass) of the same material. Other useful substrate configurations utilizing inorganic crystals and/or glasses will be apparent to those of skill in the art.

### Metals

Metals are also of use as substrates. The metal can be used as a crystal, a sheet, or a powder. The metal can be deposited onto a backing by any method known to those of skill in the art including, but not limited to, evaporative deposition, sputtering and electroless deposition.

Any metal that is chemically inert towards the detection layer may be useful as a substrate. Some suitable metals for substrates include, but are not limited to, gold, silver, platinum, palladium, nickel and copper. In some examples, more than one metal may be used. The more than one metal can be present as an alloy or metals can be formed into a layered “sandwich” structure, or metals can be laterally adjacent to one another. For example, the metal used for the substrate surface may be gold and particularly gold layered on titanium.

The metal layers can be either permeable or impermeable to materials such as liquids, solutions, vapors, and gases.

When the permeability of the substrate is not a concern and a layer of a metal film is used, the film can be as thick as is necessary for a particular application. Exemplary films are of a thickness of from about 0.01 nanometer to about 1 micrometer. In a further example, a film is of a thickness of from about 5 nanometers to about 100 nanometers. In yet another example, the film is of a thickness of from about 10 nanometers to about 50 nanometers.

### Substrate Surfaces

The structure of the surface of a substrate (or a pad on the substrate) may have an effect on the anchoring of the sensing layer which is associated with the surface of the substrate. Mechanical and/or chemical techniques may be used to engineer the surface. The surface of each of the above enumerated substrates (or pads) can be substantially smooth. Alternatively, the surface can be roughened or patterned by rubbing, etching, grooving, stretching, oblique deposition or other similar techniques known to those of skill in the art. Of particular relevance is the texture of the surface that is in contact with the fiber bundles of the detection layer.

The substrate can also be patterned using techniques such as photolithography (Kleinfield et al., J. Neurosci. 8:4098-120 (1998)), photoetching, chemical etching and microcontact printing (Kumar et al., Langmuir 10: 1498-511 (1994)). Other techniques for forming patterns on a substrate will be readily apparent to those of skill in the art.

The size and complexity of the pattern on the substrate is limited only by the resolution of the technique utilized and the purpose for which the pattern is intended. For example, using microcontact printing, features as small as 200 nm have been layered onto a substrate. See, Xia et al., G., J. Am. Chem. Soc. 117:3274-75 (1995). Similarly, using photolithography, patterns with features as small as 1 μm have been produced. See, Hickman et al., J. Vac. Sci. Technol. 12:607-16 (1994). Some patterns that are useful include those which comprise features such as wells, enclosures, partitions, recesses, inlets, outlets, channels, troughs, diffraction gratings and the like.

The patterning may be used to produce a substrate having a plurality of adjacent wells, wherein each of the wells is isolated from the other wells by a raised wall or partition and the wells do not fluidically communicate. Thus, an analyte, or other substance, placed in a particular well remains substantially confined to that well. In another example, the patterning allows the creation of channels through the device whereby an analyte can enter and/or exit the device.

The pattern can be printed directly onto the substrate or, alternatively, a “lift off” technique can be utilized. In the lift off technique, a patterned resist is laid onto the substrate, an organic layer is laid down in those areas not covered by the resist and the resist is subsequently removed. Resists appropriate for use with the substrates disclosed herein are known to those of skill in the art. See, for example, Kleinfield et al., J. Neurosci. 8:4098-120 (1998). Following removal of the photoresist, a second organic layer, having a structure different from the first organic layer can be bonded to the substrate on those areas initially covered by the resist. Using this technique, substrates with patterns having regions of different chemical characteristics can be produced. Thus, for example, a pattern having an array of adjacent wells can be created by varying the hydrophobicity/hydrophilicity, charge, and other chemical characteristics of the pattern constituents. For example, hydrophilic compounds can be confined to individual wells by patterning walls using hydrophobic materials. Similarly, positively or negatively charged compounds can be confined to wells having walls made of compounds with charges similar to those of the confined compounds. Similar substrate configurations are accessible through microprinting a layer with the desired characteristics directly onto the substrate. See, Mrkisch, M .; Whitesides, G. M., Ann. Rev. Biophys. Biomol. Struct. 25:55-78 (1996). The patterned substrate may thus control the alignment of the fiber bundles.

### Fabrication

A sensing layer or film in a sensor can be produced using several alternative coating methods. In general, the core components needed for fabrication of the sensing film include the substrate and the formulation of the film. The substrate as described above may have a metallic, glass, or ceramic surface, e.g., a pad 710 on substrate 700 of FIG. 1, for the formulation. The formulation contains a phage material and other components needed for the coating.

The formulation, in one example of the present disclosure contains an engineered phage material with additional ingredients selected according to the target analyte to be sensed. The engineered phage material may be an M13 engineered phage and may be used in solution at a concentration range between 0.01 mg/mL to 20 mg/mL (core sensing materials). One or more acid/base solvents may be used to provide the solution with a pH value range from 1 to 6, and solution may be weakly buffered, for example, with a phosphate buffer. Metal ions and nanoparticles may also be included in the solution, and typical metals used include Au, Fe, Zn, Cr, Co, Cu, Al, Ag, V, Pd, Pt (core sensing materials) depending on the target analyte. In some examples, the formulation may also contain porphyrins and/or metallic porphyrins that assist in the response against the target analyte. A polymer crosslinking agent, such as glutaraldehyde may also be added to the solution. In some examples, the formulation may also contain organic solvents (such as chloroform) during the preparation.

FIG. 8 is a flow diagram of a process 800 for manufacturing a sensing film. Process 800 begins with fabrication and preparation of a substrate with a surface suitable for coating with the sensing layer. The substrate may have a metallic surface on which the coating is to be formed or that are patterned to provide electrical traces or electrodes. The metallic surface may be a surface of a pad or layer of Au, Si, Pd, Pt. Al, Ag, or a combination of these formed on an underlying crystal such as quartz. The metal surface can be smooth, with surface roughness of less than 2 nm and generally has an area between about 0.01 mm² and 1000 mm². The surface may be a ridged, continuous, flat surface or a multi-step functional surface. In the case of a multi-step functional surface, e.g., an interdigitated electrode on a surface acoustic wave device, the step height may be between 20 nm and 10 μm. The width of each step area can be between 100 nm and 20 μm. In one example, the metallic surface is the surface of an interdigitated electrode made of gold on an underlying surface of quartz. A substrate including a glass or crystal surface on which the sensing layer is to be form may be made of or may include a pad of quartz or silicon dioxide.

Process 800 creates the formulation for the sensing layer in processes 820 and 830. The formulation may be combined in a specific order to allow aggregation or flocculation with a strong bond to create a partial suspension. Combining process 820 combines the core sensing components including the engineered phage material and the metal ions or nanoparticles. Next, solvent introduction process 830 adds the solvent to the core sensing components to adjust the pH value mixture and to disperse the core sensing materials into fine particles. The use of this solvent mixture, compared to just the core sensing materials, makes the formulation more uniform and well-dispersed, which allows stronger binding between substrate and formulation.

A pretreatment process 840 applies a treatment to the substrate surface to tune the surface charge before coating the surface with the formulation. Pretreatment process 840 may include cleaning the substrate surface with a strong oxidizing agent such as Piranha solution or etch. Pretreatment process 840 may further or alternatively include exposing the substrate surface to oxygen plasma to form a hydrophilic surface. Further, pretreatment process 840 may further or alternatively include a chemical treatment to allow a subsequent crosslinking reaction with the formulation. An example of crosslinking chemical treatment may use glutaraldehyde applied to the substrate surface. Additionally, pretreatment of the surface may include masking of portions of the substrate that are not to be cover by the sensing layer.

An application process 850 applies the formulation to the substrate surface to form the sensing layer or film. Application process 850 may employ several different application techniques including a self-templating/dip coating process, a spin coating process, a spray coating process, or a thermal evaporation process.

In self-templating/dip coating, the substrate is immersed in the formulation, and at a controlled speed with is lifted with the substrate surface typically being perpendicular to the formulation surface. This coating process be performed by dipping the surface of the substrate into the liquid formulation follow by a machine-controlled linear motion stage lifting the substrate from the formulation at a set controllable speed that may be in a range between 5 μm/min and 30 cm/min. In most situations, the slower the speed, the thicker the coating. The amount of formulation used may be between 2 mL and 2 L, only a portion of which adheres to the substrate. The lifting direction can also be at an angle between 90 degrees (perpendicular) and 45 degrees from the surface of formulation solution. The coating environment during the process may be kept at a temperature of may be between 4° C. and 40° C. and a humidity between 0% humidity and 90% relative humidity. The formulation solution can be heated or cooled through a temperature controller. The range of this heater/cooler can be between -10 degrees Celsius and 60 degrees Celsius. The film coated onto the substrate at the liquid-substrate-air interface can be further dried (or accelerated drying) through an air knife (the speed of airflow can range between 50 mL/min and 2 L/min).

For a spin coating process, the substrate may be fixed on a spin chuck under vacuum pressure, and a liquid formulation is deposited onto the substrate before or during the spinning process. The amount of formulation deposited on the substrate surface can be between 100 μL per 1000 mm² and 5 mL per 1000 mm². The method of deposition can be through an automatic precision pipette, or a formulation dispenser. The spinning speed may accelerate at between 10 rpm/s and 500 rpm/s up to be between 300 rpm and 6000 rpm. During the spin coating process, solution spun from the spinning substrate can be recollected and reapplied to the substrate, and the temperature of the coating environment may be between 4° C. and 40° C. The humidity of the coating environment may be between 0% humidity and 90% relative humidity. The rotation time of the spin coating process is generally between 20 s and 1500 s.

For a spray coating process, the formulation may be loaded in either a sprayer or a container connected to an air knife. Air pressure, e.g., between 5 psi and 60 psi, is applied onto the formulation and is forced through the sprayer or air knife and out toward the substrate to generate formulation aerosols with a diameter ranging from 0.01 μm to 50 μm. The distance from the sprayer/air knife to the substrate can range from 2 cm to 60 cm, and the amount of formulation per application can range from 100 μL per 1000 mm² of substrate surface to 2 mL per 1000 mm² of substrate surface. The coating environment may be at a temperature between 4° C. and 40° C. and a humidity between 0% humidity and 90% relative humidity.

Of the three application processes, dip coating may provide the most highly organized structure for fibers in the sensing layer. Such organization may be needed for consistent color variation in response to a target analyte. Spin coating and spay coating may result in a less organized structure, which may not provide consistent color performance but that provides efficient capture to change acoustic characteristics as needed, for example, in QCM or SAW sensors. Any the three coating processes may be repeated multiple times to increase the thickness of a sensing layer. The desired range of film thickness is typically between 20 nm and 10 μm.

A finishing process 860 of process 800 can postprocess the sensing layer coated onto a substrate. Finishing process 860 may particularly include crosslinking of the material in the layer and application of a porous protective layer. An application of crosslinking solution, e.g., a solution of glutaraldehyde, can chemically crosslink the resulting thin film. The crosslinking solution may be applied either through vaporization onto the thin sensing film, or the sensing film may be washed with the crosslinking solution. A thin protective layer, which is a semi-porous layer, may be deposited on top of the film, e.g., through the chemical vapor deposition process, to stabilizes or protect the film but still allow the target analyte to reach the film and activate receptors in the film. The protective layer can be made of materials such as silicon dioxide, titanium dioxide, or aluminum oxide.

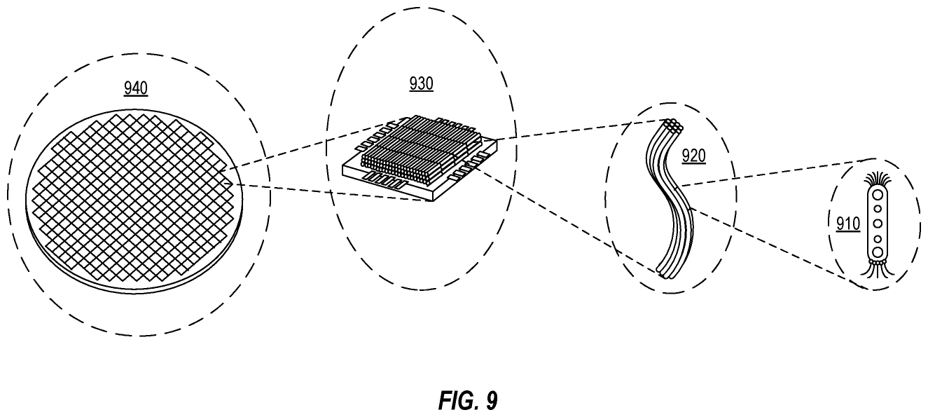

FIG. 9 illustrates a hierarchy of structures in examples of compact or miniature sensors capable of sensing one or more specific chemical compounds using bioengineered phage technology. FIG. 9 particularly shows a M13 phage 910 that is bioengineered using known techniques to interact with a target analyte to be measured. Multiple phages 910 are assembled onto phage nanofibers or bundles 920, which are used in the coating on a sensor chip 930 for sensing the target analyte associated with the bioengineered phage 910. Many sensor chips 930 can be part of a wafer 940 and simultaneously processed to produce a batch of sensors for sensing of the same analyte.

FIG. 10 shows a block diagram of sensor system 1000 including an array 10 of different sensor chips 10-1 to 10-N with different M13 phages targeting different analytes. Sensor system 1000 includes an electronic sensing module 20 that uses sensor chip array 10 to sense an assortment of different analytes, and measurements output from different combinations of measured analytes from sensor chips 10-1 to 10-N may indicate a signature that electronic sensing module 20 can associate with a particular condition such as a complex aroma, e.g., the aroma of a particular type of food. Fabrication processes and sensor chips in accordance with examples disclosed herein may permit a complex sensor such as shown in FIG. 10 to be inexpensively produced while still providing complex sensing capabilities that are difficult to achieve using prior sensing systems.

Although specific implementations have been disclosed. these implementations are only examples and should not be considered as limitations. Various adaptations and combinations of features of the disclosed implementations are within the scope of the following claims.

What is claimed is:

1. A sensing system comprising a sensor chip that includes:
a substrate having a sensing area;
a coating on the sensing area, the coating containing fiber bundles including a phage with receptors that interact with a target analyte;
a first pair of interdigitated electrodes on the substrate on a first side of the sensing area; and
a second pair of interdigitated electrodes on the substrate on a second side of the sensing area, the second side being opposite the first side.

2. The sensing system of claim 1, further comprising a driver circuit connected to the first pair of interdigitated electrodes, the driver circuit applying to the first pair of interdigitated electrodes a drive signal that creates acoustic vibrations in the substrate.

3. The sensing system of claim 2, further comprising a sensing circuit connected to the second pair of interdigitated electrodes, the sensing circuit measuring changes that the interaction of the coating with the target analyte cause in acoustic vibrations.

4. The sensing system of claim 3, further comprising a control circuit receiving from the sensing circuit measurements of the changes, the control circuit using calibration data to generate a measurement signal indicating a concentration of the target analyte.

5. The sensing system of claim 3, wherein the pad area contains a quartz pad on which the coating resides, and the acoustic vibrations comprise resonant vibration in the sensor chip.

6. The sensing system of claim 3, wherein the sensing circuit measures surface acoustic waves at a surface on which the coating resides

7. The sensing system of claim 3, measure an electric signal that vibrations in the substrate induce in the second pair of interdigitated electrodes.

8. The sensing system of claim 1, wherein the sensing system is one of a quartz crystal microbalance (QCM) senor or a surface acoustic wave (SAW) sensor that includes the sensor chip.

9. The sensing system of claim 1, wherein the substrate comprises a piezoelectric material.

10. The sensing system of claim 1, wherein the piezoelectric material is quartz.

11. A method for fabricating a sensor chip comprising:
preparing a surface in a sensing area of a substrate of the sensor chip;
a combining core sensing materials including a phage and receptor material in a solution; and
coating the sensing area with the solution using a coating process selected from:
dispensing the solution onto the sensing area and spinning the sensor chip to distribute the solution across the sensing area; and
a spraying the solution onto the sensing area.

12. The process of claim 11, further comprising including in the solution an acid that causes flocculation to create a partial suspension of the core sensing materials.

13. The method of claim 11, wherein preparing the surface in the sensing area comprises forming a gold pad covering the sensing area.

14. The method of clam 13, wherein the substrate comprises a quartz substrate.

15. The method of claim 11, further comprising applying a crosslinking agent to the solution on the sensing area to crosslink bundles of the phage and create a structure holding the receptor material.
